(19) United States
(12) Patent Application Publication
(10) Pub. No.: US 2022/0151701 A1
(43) Pub. Date: May 19, 2022

(54) METHODS FOR REALISTIC AND EFFICIENT SIMULATION OF MOVING OBJECTS

(71) Applicant: VIRTAMED AG, Schlieren (CH)

(72) Inventors: Carlota SOLER ARASANZ, Schlieren (CH); Tobias MARTIN, Schlieren (CH)

(73) Assignee: VIRTAMED AG, Schlieren (CH)

(21) Appl. No.: 17/592,937

(22) Filed: Feb. 4, 2022

Related U.S. Application Data
(63) Continuation-in-part of application No. 16/155,227, filed on Oct. 9, 2018, now abandoned.
(60) Provisional application No. 62/696,426, filed on Jul. 11, 2018.
(60) Provisional application No. 62/569,768, filed on Oct. 9, 2017.

(51) Int. Cl. A61B 34/10; A61B 34/20; A61B 17/04

(52) U.S. Cl. CPC A61B 34/10; A61B 34/20; A61B 17/0401; A61B 2034/108; A61B 2034/107; A61B 2034/2046; A61B 2034/104

(57) ABSTRACT

A method is proposed to simulate a moving rod with a changing orientation such as bending or twisting in a real-time surgical threading simulation application. A Projective Dynamics local-global solver is adapted with discretized Cosserat object position and orientation constraints and potentials for the local and global solving steps. Rotatable rigid or deformable bodies may be simulated accordingly, such as for instance rods, with potential weights accounting for the material parameters and geometric properties of the objects, such as the radius, the mass density, the length, and/or, for elastic thin objects, the Young's modulus, thus enabling realistic simulation for different values. Furthermore, the proposed methods converge after a small number of iterations, independently from the mesh resolution, enabling fast implementation in a diversity of computer graphics simulation applications.

### CROSS-REFERENCE TO RELATED APPLICATION

This application is a continuation-in-part of U.S. patent application Ser. No. 16/155,227, filed on Oct. 9, 2018 which claims the benefits of U.S. Provisional Application No. 62/696,426, filed on Jul. 11, 2018, and U.S. Provisional Application No. 62/569,768, filed on Oct. 9, 2017. The entire contents of which are hereby incorporated by reference within their entireties.

### FIELD OF THE INVENTION

The present disclosure applies generally to computer science and more specifically to computer graphics simulation of moving objects with a changing orientation, such as for instance twisting rods.

### BACKGROUND

Computer-generated imagery, also known as computer graphics (CG) aims at digitally representing visual content as an alternative or a complement to real images. Computer graphics have a diversity of applications in various fields such as the production of entertainment software, movie animations, video games, advertising material, as well as virtual reality simulation and training applications. In the latter interactive applications, there is a need to generate more realistic computer graphics images with more efficient computing resources, so as to improve the end user experience while minimizing the costs to produce the interactive graphics. In that context, recent research has focused on more efficient and more realistic methods to simulate rigid or deformational objects with a changing orientation and in particular rods, such as bending and twisting hair in character animation, or hair, blood vessels, threads, catheters and suturing in medical simulators.

### Surgical Suturing Simulation

The acquisition of surgery skills requires to master a number of suturing procedures, in particular in minimally invasive operations under endoscopy. U.S. Pat. No. 4,493,323 describes an exemplary arthroscopic system for knee repair which involves the manipulation of tools holding needles for suturing a diversity of tissues in the knee joint under endoscopy control. In general, the needles are forced through the tissue to be sutured at contact points, and the thread follows the complex motion of the needle around the contact points.

Learning how to manipulate the surgical instruments, including the needle holder for suturing, is therefore a key skill for apprentice surgeons to acquire. Surgical training therefore involves more and more mixed reality medical simulators comprising replicates of the body components, such as anatomy models, and replicates of the surgical tools, such as a haptic feedback system arrangement as described for instance in U.S. Pat. No. 8,956,165, or using real instruments replica arranged with sensors as described for instance in U.S. Pat. No. 8,992,230, in combination with a virtual reality simulation of the endoscopy scene.

In order to provide as realistic as possible simulation to the trainees, such simulators need to calculate and render in real time on the display screen all the virtual objects involved in the suturing procedures. These objects comprise the virtual models of the anatomy components and the surgical tools as tracked with sensors to adapt in real time to the trainee manipulation, as well as elements such as the virtual suture contact points and the virtual thread which only exists in the abstract, virtual reality environment without a physical counterpart.

The visual quality of this rendering is therefore key for the trainee to properly exercise his/her eye-hand coordination when manipulating the needle holder instrument under simulated endoscopy control when learning a suturing procedure. Most prior art solutions such as the one disclosed in U.S. Pat. No. 8,956,165 use finite element modeling (FEM) such as for instance mass-spring models to simulate the motion and collisions of the surgical thread with the anatomy model tissues during suturing. In general, the surgical thread may be modeled as a rod made of a series of connected simple cylindrical object models. The real-time calculation of such models is computationally expensive and requires a tradeoff between the cost of the simulator hardware and the realism of the procedures to be rendered, for instance by using only a limited number of elements for the rod or not fully representing the complex twisting and bending motion as observed with a real thread under endoscopy. There is therefore a need for improved computer graphics and simulation models for the surgical threads in a number of suturing procedures.

### Rod Models

Rod simulation is a difficult problem since both positions and orientations on the rod need to be predicted. Indeed, simulating rods solely with positions yields a simplistic and unrealistic behavior. Further tracking the orientation along the rod enables to simulate twisting and torsional effects. Therefore, in order to reproduce physically plausible rod behavior, it is essential to employ a fundamental theory that models twists, as done by Cosserat theory. Cosserat theory equips points of the material with an orientation. This enables to reproduce how a rod stretches to some material and how a twist propagates along the rod when a rotational force is applied.

Early theories date from around 1859, Kirchhoff [Kir59] being one of the first to devise a three-dimensional theory that replaced the 1D-body approach. His early theory and further research [Dil92] led to an explicit representation of the rod's center-line. The orientation of the rod is represented by several material frames, which enable to keep track of twisting and bending. A contemporary example of Kirchhoff rod simulation is discrete elastic rods [BWR*08, BAV*10], where the material frame is treated as quasi-static by assuming an inextensible rod. Later theories involving the Cosserat brothers (1909) led to the formulation of Cosserat rods [Rub13]. This theory models the rod as a space curve with two additional directions, which model material fibers in the cross-section of the rod. These fibers can stretch in length and shear relative to the normal of the cross-section and the tangent of the space curve, which allows to simulate extensible rods and hence provide a broader model compared to Kirchhoff rods.

Pai et al. [Pai02] was the first to introduce the Cosserat model to the computer graphics community with an implicit representation of the rods, aimed at simulating threads and catheters in virtual surgery procedures like laparoscopy. In this method, the centerline is expressed implicitly by an approximation of smooth curves, which makes collision detection difficult.

Explicit discretization of Cosserat rods is a more convenient approach, as the geometry of the rod can be easily reconstructed. The CORDE method [ST07] uses a deformation model for simulating dynamic elastic rods based on the Cosserat theory with continuous energies. After discretizing the rod, the energy is computed per element with finite element methods, and thus the dynamic evolution of the rod is obtained by numerical integration of the resulting La-grange equations of motion. Although the results are shown to be physically plausible, the explicit time integration of this approach requires very small time steps and strong damping to remain stable, which is the main performance bottleneck of the simulation.

Lang et al. [LLA11] introduced a geometric model for discretizing the rod similar to the one in CORDE [ST07] and derives the equations of motion in the continuous domain by applying Lagrangian field theory. These equations are solved using the finite difference method together with standard solvers for stiff differential equations. Casati et al. [CBD13] presented an integration scheme based on power expansions, which reaches higher precision faster compared to classical numerical integrators. Their method is based on a semi-implicit time stepping scheme, which is by definition less stable than an implicit integrator, and hence the motivation to simulate rods with an implicit scheme such as Projective Dynamics.

### Rod Simulator Methods—PBD, FEZ, and PD

Position-based dynamics methods have become popular in recent years as simple and fast methods to simulate elastic rods as well as cloth, volume deformable bodies, rigid body systems and fluids, as reviewed by Bender et al. in “A survey on Position-Based Dynamics, 2017”, Eurographics 2017. In particular, previous methods to simulate stable Cosserat rods with position based dynamics (PBD) have been recently disclosed [KS16, DKWB18].

These PBD methods however have some inherent limitations in accuracy and robustness especially under severe constraints. Moreover, they do not support adaptive meshing, as changing the mesh resolution results in a different physical behavior.

In contrast to PBD methods, Finite element methods (FEM) implementations as described for instance in [BWR*08, BAV*10] provide accurate simulations thanks to the internal/external force simulation, but they require small time steps to ensure stability, making them too computationally expensive to implement in practice for most simulators.

As a significant improvement over PBD methods for more realistic and more robust simulations, the Projective Dynamics solver introduced by Bouaziz et al. in “Projective Dynamics: Fusing constraint projections for fast simulation”, CM Trans. Graph. 33, 4 (July 2014), 154:1-154:11 [BML*14] combines the simplicity and performance of Position-Based Dynamics simulations with the accuracy and robustness of an Implicit Euler solver, even under extreme deformation constraints. This Implicit Euler solver method is based on a local-global constrained optimization which efficiently applies to both real-time and offline simulation in many different computer graphics applications. The local steps use a set of local constraints and may thus be parallelized as small independent optimization problems on the Graphical Processing Units (GPUs) of recent computing devices hardware architectures, thus enabling efficient enough solving for real-time rendering applications of computer graphics simulations. By proper discretization of the energy constraints, the solver is also robust against non-uniform meshing with different resolutions. In certain applications, a fixed set of constraints may also be pre-defined so that the linear system of the global step can be pre-factored. In other applications, the global solver step may also be computed on-the-fly, for instance in accordance with dynamic interaction from the end user in an interactive system setup, and the global solution step may be executed as well.

However, the Projective Dynamics method as initially described by Bouaziz et al. still suffers from certain limitations when applied to the simulation of the motion for rigid and/or deformational objects with a changing orientation, such as for instance rods. In particular, while the prior art Projective Dynamics solver assumes a mesh consisting of vertices with 3D positions and linear velocities, thus supporting the estimation of the linear velocity as part of the linear momentum of moving objects, its mathematical modeling does not support orientations and angular velocities. Thus it does not preserve the angular momentum as required with twisting and bending rods, the angular momentum comprising an angular velocity and a rotational external force (torque) in addition to the object linear velocity. More generally, the Projective Dynamics method does not able to simulate complex phenomena such as the forming of plectonemes, which can be observed when twisting a rod or a volumetric object.

There is therefore a need for improved Projective Dynamics solver methods which also allow to more realistically and efficiently simulate a broader range of moving objects with different material and geometrical parameters.

### SUMMARY

In a possible embodiment, a computer graphics method is disclosed to render, with a processor, a twisting and bending surgical thread in a medical simulator, wherein said method comprises:
Acquiring in real time the position and orientation of a needle holder instrument replica and the position and orientation of an endoscope replica from one or more sensors tracking the manipulation of the instrument and the endoscope by the end user;
Detecting, from the tracked position and orientation of the needle holder, a collision between a simulated needle and an anatomy tissue model in a virtual environment;
Creating, at the collision point, a suture fixation point in the virtual environment;
Determining a set of constraints applicable to a virtual surgical thread at the suture fixation point, wherein the virtual surgical thread is represented in the virtual environment as a rod attached to the suture fixation point, wherein the set of constraints comprise at least a bend and twist constraint which depends on the geometrical and material properties of the rod model and a suture fixation constraint which depends on the physical properties of the anatomy tissue model at the suture fixation point, to constrain the suture to slide through the suture fixation point if the tracked position and orientation of the needle holder indicates that the user is pulling the suture from the anatomy tissue model at the suture fixation point;
Discretizing and modelling the rod as a Cosserat rod with one or more rod elements;
Estimating, with a local-global solver, the position and orientation of each rod element using the set of constraints;
Determining, from the position and orientation of the needle holder instrument replica and the position and orientation of the endoscope replica, which elements of the rod are visible in the scene to be rendered;
Rendering each visible rod element in the computer graphics simulation.

In a possible embodiment, a computer graphics method is disclosed to render, with a processor, a moving object, wherein said method comprises:
discretizing the object into a plurality of elements;
identifying, for each of the plurality of elements, a current position and orientation, and a current linear velocity and a current angular velocity corresponding to an object motion to simulate;
estimating, for each of the plurality of elements, a predicted position and orientation as a function of the current position, orientation, linear velocity and angular velocity;
formulating, for each of the plurality of elements, object motion constraints Cᵢ corresponding to the object motion to simulate;
for each of the plurality of elements, projecting, with a local solver, the predicted element position and orientation into auxiliary projection variables pᵢ on the object motion constraints Cᵢ;
estimating, with a global linear system solver, a refined predicted position and orientation for each of the plurality of elements of the moving object as a function of the auxiliary projection variables pᵢ for each of the plurality of elements and of the material and/or geometrical properties of the moving object; and
rendering each of the plurality of elements of the moving object at the respective refined predicted positions in the computer graphics simulation.

In a possible embodiment, the local and global solver steps may be iterated several times.

In a possible embodiment, discretizing the object may comprise modeling the object as a Cosserat object with one or more elements. The Cosserat object elements may then be associated with discretized position variables x∈R³ and discretized Cosserat object orientation quaternion variables u∈R⁴.

In a possible embodiment, the object motion constraints Cᵢ may comprise a Cosserat object bend and twist constraint C_{BT} as a function of a twist strain defined for each object element, and the local solver projection on the Cosserat object bend and twist constraint C_{BT} may be optimized when the relative curvature between any pair of adjacent element orientations is zero.

In a possible embodiment, the object motion constraints Cᵢ may comprise a Cosserat object stretch and shear constraint C_{SE} as a function of a stretch strain defined for each object element. In a further possible embodiment, the local solver projection on the Cosserat object stretch and shear constraint C_{SE} may comprise a first local optimization step, with the local solver, on the position variables and a second local optimization step, with the local solver, on the orientation variables, the first and the second steps being independent from each other. The solution to the first local optimization on the position variables may be reached when the element's differential positions have a unit length and are aligned with the normal of the Cosserat object's cross section, so as to preserve the element's length as in its initial configuration. The solution to the second local optimization on the orientation variables may be reached when the rotational difference between the normal of the Cosserat object's cross section and the tangent of the element is minimal.

In a possible embodiment, predicting, with a global solver, the motion of the Cosserat object may comprise calculating a matrix of weighted Cosserat potentials, the potentials being calculated as a function of the auxiliary projection variables pᵢ for the plurality of elements, and the weights being calculated as a function of the material and/or the geometrical properties of the Cosserat object.

In a possible embodiment, the Cosserat object may be a Cosserat rod. The geometrical properties of the Cosserat rod may defined by at least one or more of the following parameters: the radius of the rod and/or the length of the rod. The material properties of the Cosserat rod may be defined by at least the mass density of the rod material. In a further possible embodiment, the rod may be elastic and the material properties of the Cosserat rod may be further defined by the Young's modulus of the rod.

In possible alternate embodiments, the Cosserat object is a Cosserat shell, a Cosserat volume, or the object may be rigid and the Cosserat object may be modeled as a Cosserat point.

In a possible embodiment, a predicted linear velocity may be calculated as a function of the current position and the refined predicted position for each element, a predicted angular velocity may be calculated as a function of the current orientation and the refined predicted orientation for each element, and the predicted linear velocity, the predicted angular velocity and the predicted refined predicted position and orientation may be used as the current estimates for each of the plurality of elements of the moving object in the a next rendering calculation.

In a possible embodiment, the twisting or bending rod is any object selected from the group consisting of a surgical thread, a suturing thread, a blood vessel, and a strand of hair.

In a possible embodiment, the suture fixation constraint is formulated to minimize a set of elementary constraints such as a constraint on the position of the suture fixation, a constraint on the orientation of the suture fixation, a constraint to ensure that the suture preserves its rest length, and/or a pulling constraint on the fixation tissue using the suture's position at the suture fixation as target location from the pulling.

In a possible embodiment, at least one of the suture fixation elementary constraints is parametrized using barycentric coordinates according to the placement of the suture fixation along the Cosserat rod element.

### LIST OF FIGURES

FIG. 1A represents an exemplary rod simulator system and FIG. 1B depicts an exemplary computer graphics workflow according to certain embodiments of the present disclosure.

FIG. 2 illustrates a possible modelling of a Cosserat rod, defined by an arc length parametrized curve, where each point is augmented by an orientation.

FIG. 3 illustrates a possible Cosserat rod discretization with points, elements and orientations representing the material frames.

FIG. 4 describes a possible algorithm to extend a Projective Dynamics solver with preservation of the angular momentum so that the rod orientations are tracked in addition to the positions.

FIGS. 5A-5B illustrate the rendering results of a first experiment at three time snapshots of motion simulations of a hanging rod attached on both ends, under the gravitational force and for different mesh resolutions, comparing the proposed method with a prior art PBD method.

FIGS. 6A-B illustrate the rendering results of a second experiment at three time snapshots of motion simulations of a hanging rod, attached only on one hand, under the gravitational force and for different mesh resolutions, comparing the proposed method with a prior art PBD method.

FIG. 7, FIG. 8A, FIG. 8B and FIG. 9 illustrate the rendering results of further experiments comparing the proposed method with the results of a high-resolution FEM simulation.

FIG. 10 further illustrate the rendering results of an out-of-plane curl effect simulation experiment comparing the proposed method with a prior art PBD method.

FIG. 11 compares a real rod to the rendering simulations with the proposed method, using the same geometric and material parameters.

FIG. 12A and FIG. 12B represent respectively a 3D and a 2D view of suture fixation points along a Cosserat rod.

FIG. 12C represents a Cosserat element with a suture fixation point on the element segment, which introduces an extra constraint to the calculation of its rendering.

### DETAILED DESCRIPTION

### Rod Simulator System

In various possible embodiments, these methods may be computer-implemented and executed by a computer graphics generator system in a computer gaming setup, a computer-aided design application, in an entertainment media content generator tool, or in a professional practice simulator, for instance a medical or a surgery training simulator.

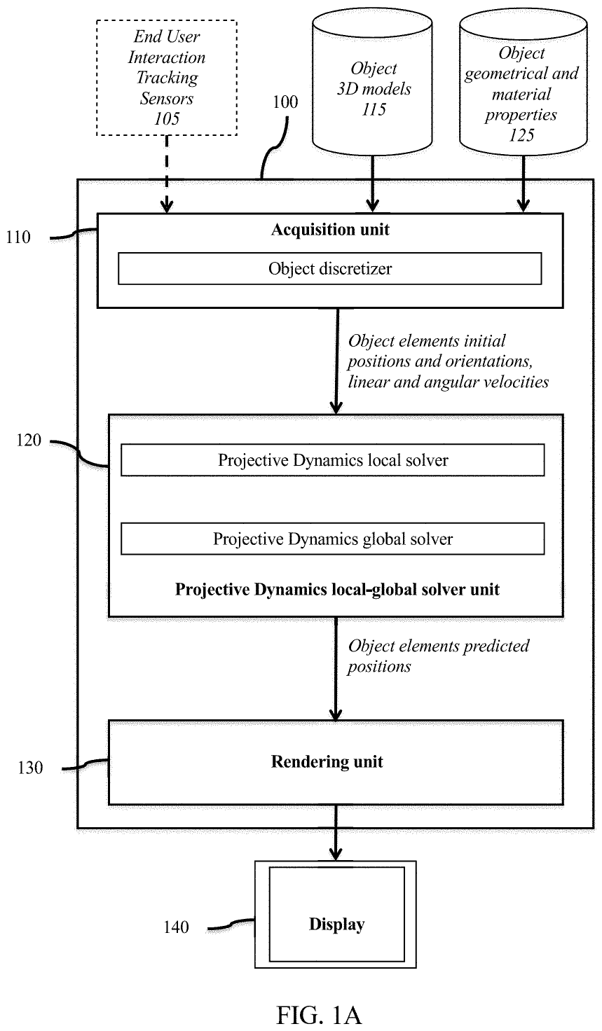

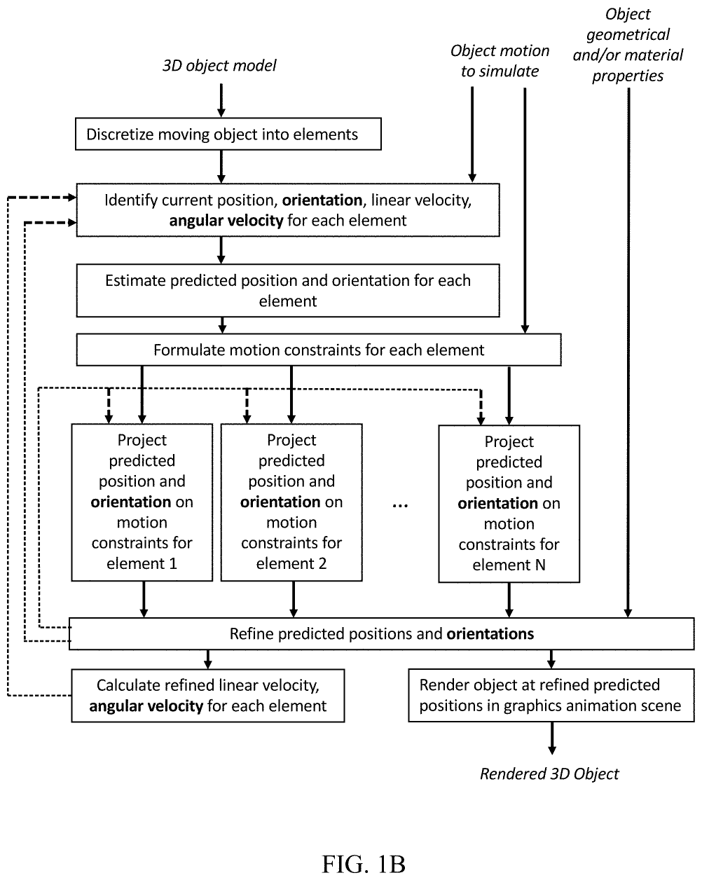

The computer graphics generator system can include hardware and/or software elements configured for simulating one or more computer-generated objects. Simulation can include determining motion and position of an object over time in response to one or more simulated forces or conditions. The computer graphics generator system may be invoked by or used interactively by a user of the one or more computers and/or automatically invoked by or used by one or more processes associated with the one or more computers. In a possible embodiment, as represented on FIG. 1A, the computer graphics generator system 100 may be part of a virtual reality or augmented reality setup configured with tracking sensors 105 to interact with an end-user in real-time. Position and orientation information of the end-user and/or of an instrument manipulated by the end-user may be tracked and combined with graphical models of 3D objects 115 in accordance with their physical properties 125 ((for instance geometrical and material properties) to simulate and render a computer-generated scene (not represented) comprising the simulated moving object.

The computer graphics generator system 100 may for instance by part of a medical training simulator setup as described for instance in U.S. Pat. No. 8,992,230 or PCT patent application WO2017064249, both in the name of Virtamed AG. In such a setup, the computer graphics generator system 100 may combine 3D visual models 115 of various objects such as organs and tools, for instance a virtual suturing needle, with their predefined physical properties, such as geometrical and material properties 125, for instance replicating the properties of a real surgical thread, to realistically simulate the behavior of the moving rod in accordance with the tracked end user gesture as tracked from the sensors 105, for instance a sewing gesture.

As will be apparent to those skilled in the art of computer graphics simulation, computer graphics generator system 100 may derive from the 3D models 115 and the object geometrical and material properties 125 various constraints to calculate in real-time the moving object position and orientation and render it accordingly with a rendering unit 130 as part of an animated graphics scene. The rendered scene may then be displayed on a screen 140.

The computer graphics generator system 100 may comprise an acquisition unit 110 to acquire the object position and orientation from the tracked user interaction as measured by the sensors 105. In other embodiments (not represented), the acquisition unit 110 may acquire the object position and orientation in accordance with predefined scene requirements, for instance in a predefined entertainment or gaming animation application or surgical threading simulation.

In surgical threading, the needle is forced through the tissue to be sutured at contact points. For a more robust simulation of a suturing scenario in the virtual environment, the computer graphics generator system 100 may identify a suture fixation point at the contact point where the rod is attached to the tissue. The computer graphics generator system 100 may further define a suture fixation constraint applicable to the suture fixation so that user manipulation may be more realistically rendered by the simulator, in particular when the user is pulling the rod away from the tissue it is attached to, as part of the sewing gesture. The suture rod will then be constrained to pass through the suture fixation point within the tissue and slide through it if the user pulls from the needle to suture the tissue. As will be apparent to those skilled in the art, improvements to the prior art methods of computer graphics simulation are therefore needed, to account for the combination of the complex motion of the needle with a certain amount of twisting and bending of the rod together with additional suture fixation constraints. The acquisition unit may further comprise an object discretizer to facilitate the digital computation of the object motion in the computer graphics scene. In a preferred embodiment, the object may be modeled as a Cosserat object and its orientation may be represented by a quaternion, but other embodiments are also possible, for instance with rotation matrices.

In a preferred embodiment, the computer graphics generator system 100 may further comprise a local-global solver unit 120 to calculate in real-time the simulated object position and orientation in the scene in accordance with the object motion constraints in the computer graphics scene to be rendered, as well as with the object geometrical and/or material properties for increased physical realism. In a preferred embodiment, an implicit Euler solver such as the Projective Dynamics solver may be adapted to minimize Cosserat constraints, but other embodiments are also possible. The local-global solver unit 120 may apply various iterations of local and global solving to predict and render, with the rendering unit 130, the moving object elements positions in the computer graphics scene with more visual accuracy and physical realism than prior art real-time computer graphics units, as described in further details throughout this disclosure.

FIG. 1B depicts a workflow for a possible embodiment of the proposed method to simulate and render, with a computer graphics processor system 100, the motion of an object in a graphics animation scene. The method may comprise, at a current time step:
discretizing the 3D object model into a plurality of finite elements;
at a first time step, identifying the current position, orientation, linear velocity and angular velocity corresponding to the object motion to simulate for each element of the discretized object;
estimating a predicted position and orientation for each element of the moving object;
formulating motion constraints corresponding to the object motion to simulate for each element of the moving object;
projecting the predicted position and orientation on the motion constraints for each element of the moving object, with a local solver;
refining the predicted position and orientation for all elements of the moving object as a function of the object material and/or geometrical properties, with a global solver;
optionally, repeating the local and global solver minimization steps;
rendering the moving object at the refined predicted position in the graphics animation scene;
calculating a refined predicted linear velocity and angular velocity for each finite element of the moving object, and using them as well as the refined predicted position and orientation in a next rendering time step.

As will be apparent to those skilled in the art of computer graphics, the method may be iterated at different time steps in accordance with the rendering requirements for the graphics animation scene, such as the frame rate necessary to ensure the visual and physical realism of the rendered scene. The local solver steps may be executed independently for each element, and possibly separately on the positions and the orientations of each element, so as to facilitate efficient parallel implementation of the computer graphics simulation method, for instance on a GPU. In a preferred embodiment, the global solver may comprise a linear system solver which also facilitates fast simulation of the object motion. In a possible further embodiment (not illustrated) the global solver may separately solve linear equation systems respectively on the predicted position variables and the predicted orientation variables.

The proposed method is particularly suitable for simulating moving objects with changing orientations and angular velocities, such as for instance rods in the exemplary application of a surgical simulator 100. The inclusion of orientations as system variables in a Projective Dynamics solver also enables the simulation of various rotatable bodies, including rigid as well as deformable bodies. In a possible embodiment, by defining a stretch and shear constraint, which relates both position and orientation variables in the system, it is possible to simulate rotating rigid as well as deformable bodies with a Projective Dynamics solver. Such non-linear deformations, for instance attachments, can be formulated similar to an edge which preserves the length and is additionally rotated.

In a possible further embodiment, 3D object bodies simulated or animated with Projective Dynamics (or position-based dynamics) may be manipulated by adding additional soft and/or hard constraints to the basic Cosserat constraint set. For example, [BML*14] describes a handler constraint as a positional constraint that may be applied for instance to account for collisions, tissue deformation and compression, rod stretching, etc, in relation with the simulation of attachments to the 3D object bodies. In particular, positional constraints may be applied by the local-global solver to account for the non-linear deformations introduced by attachments between 3D object bodies, such as suture fixations in a surgical simulator.

For instance, in the context of Cosserat rods, to simulate the motion with a specific degree of freedom to a specified position (by using any form of tracked user input such as a computer mouse or a surgical tool sensor 105), a positional constraint, such as a handle constraint or a suture fixation constraint, may be added to the basic set of Cosserat constraints. In a preferred embodiment, a general suture fixation constraint may be formulated to minimize a set of elementary constraints such as a constraint on the position of the suture fixation, a constraint on the orientation of the suture fixation, a constraint to ensure that the suture preserves its rest length, and/or a pulling constraint on the fixation tissue using the suture's position at the suture fixation as target location from the pulling. Such a suture fixation constraint enables to fix the suture (like positional constraints). Moreover, when pulling from one endpoint, for instance in accordance with the sewing gesture as tracked from the end user manipulation of the surgical simulator instruments, the suture will be forced to slide through the suture fixation point, thus simulating a needle eye behavior. Preferably, the suture fixation constraint may depend upon the physical properties of the 3D anatomy model tissue to which the rod is sutured at the suture fixation point. Physical properties may comprise for instance material properties, for instance deformation and/or friction properties.

This suture fixation constraint essentially enforces to minimize the Euclidean distance between p and q, where p is the current position of the degree of freedom, and q is the target position in accordance with the simulation scenario. Note that other constraints may already act on this degree of freedom, so the Projective Dynamics solver can find a compromise between all these “soft” constraints and compute a position for the respective degree of freedom, which satisfies all the constraints in a least-square sense. In a further possible embodiment, the respective constraint may be further weighted with a pre-defined value, to give the soft constraints a smaller or bigger impact into the simulated solution. As will be apparent to those skilled in the art of physics-based simulation, in a further possible embodiment, for Cosserat rods, “hard” constraints may also be defined and applied to the solver, similar to FEM (Finite Element Method) practice.

### Cosserat Rods Modelling

Cosserat rods may be described by an arc-length parametrization r(s): [0,L]→R³. Every point of r(s) is associated with a frame of orthonormal vectors {d₁(s), d₂(s), d₃(s)}, also called directors. The cross-section of the rod is spanned by the directors {d₁(s), d₂(s)}. Their cross product d₃(s)=d₂(s)×d₁(s) defines the normal of the cross-section. Each orthonormal frame, also called material frame, can be represented by a single quaternion u(s). This orientation information enables to formulate energy densities for the moving object, such as for instance stretching, shearing, bending and twisting degrees of freedom.

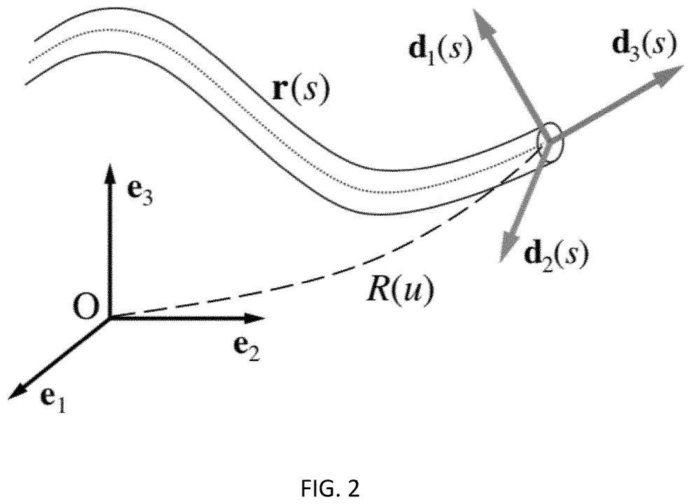

Given a fixed coordinate basis {e₁,e₂,e₃}, each director is described as dₖ=R(u) eₖ=u eₖ u, which is the quaternion rotation (denoted by R(u)) of the basis vector eₖ by quaternion u, as illustrated by FIG. 2. Note that throughout the present disclosure, we omit the parameter s for clearer notation whenever possible.

The Cosserat continuous stretch and shear potential may then be defined by the following integral, as in [LLA11] (Eq.1):

v_{SE}=½∫₀^{L}\tilde{Γ}^{T}C^{Γ}\tilde{Γ}ds

where the strain measure \tilde{Γ}∈R³. is defined in material frame coordinates as (Eq.2):

\tilde{Γ}=R(u)^{T}∂ₛr−e₃ and Γ=∂ₛr−d₃

is an equivalent expression to measure stretch and shear deformations. The tangent ∂ₛr is the spatial derivative of the centerline at a given point s and d₃ is the cross-section normal as defined above. The rod is subject to shear deformation if the direction of the tangent differs from the cross-section normal, ∂ₛr≠d₃. The rod is subject to stretch if the tangent is not unit length: ∥∂ₛr∥≠1, i.e., its length changes compared to the initial state.

The Cosserat continuous bend and twist potential may be defined as (Eq.3)

v_{BT}=½∫₀^{L}Ω^{T}C^{Ω}Ωds

where Ω denotes the material curvature vector for a given point s, which measures the rate of change in curvature as in [LLA11], i.e., (Eq.4):

Ω=R(u)^{T}∂ₛR(u) or Ω=2ū∘∂ₛu

The material curvature vector can also be formulated with the quaternion product (denoted by ∘) in Eq.4, measuring the relative rotation between the material frame orientation and its spatial derivative.

The matrices (Eq.5)

$${C}^{Γ} = ( \begin{matrix}{GA}_{1} & & \\ & {GA}_{2} & \\ & & {EA}_{3}\end{matrix} ) and {C}^{Ω} = ( \begin{matrix}{EJ}_{1} & & \\ & {EJ}_{2} & \\ & & {GJ}_{3}\end{matrix} )$$

encode the weight constants of the potential energies in terms of the cross-section area components A₁, A₂ and the cross-section geometrical moments of inertia J₁, J₂, J₃ (as in [Sim85]). In the following we assume a circular cross-section, i.e., A₁=A₂=A₃=πr² and J₁=J₂. Expressing J₁=∫∫_{A} x2 d(x,y) in polar coordinates and substituting d(x,y)=\tilde{r}d(θ,\tilde{r}) leads to the expression (Eq.6):

$${J}_{1} = {J}_{2} = {\int }_{0}^{r} {\int }_{0}^{2 π} {( \tilde{r} cos θ )}^{2} \tilde{r} d θ d \tilde{r} = \frac{π {r}^{4}}{4}$$

Finally, J₃ corresponds to the polar moment (Eq.7):

$${J}_{3} = {J}_{1} + {J}_{2} = \frac{π {r}^{4}}{2}$$

The constants E, G>0 denote the Young and shear moduli of the material, respectively.

$$G = \frac{E}{2 ( 1 + v )} ,$$

where τ is the Poisson's ratio.

### Object Discretization

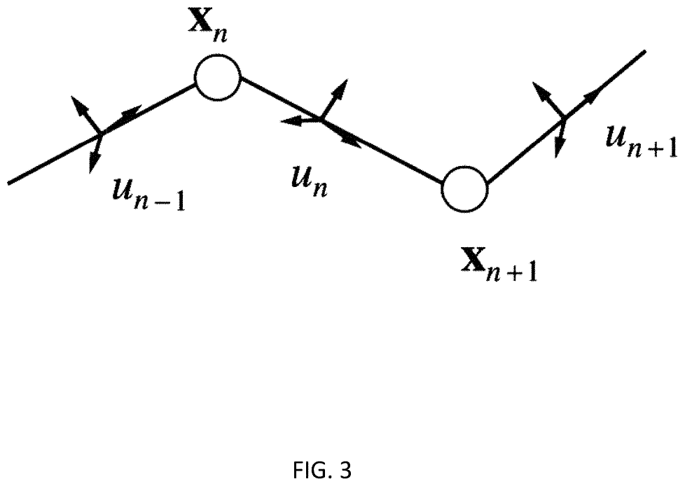

For deformable objects made of different elements each possibly with a different position and orientation, the object may preferably be discretized prior to applying the solver method. To discretize the Cosserat theory, the Cosserat object may be discretized into a set of finite elements. In the case of a rod, the Cosserat object may be uniformly sampled to obtain a piecewise linear curve with N points. Each element of the object may then be defined by two points {xₙ, xₙ₊₁}, which represent the positions of the element end points, and one quaternion uₙ, which represents the orientation of the material frame, as illustrated by FIG. 3. Hence, the sampled object consists of N−1 elements and N−1 quaternions.

The discrete stretch and shear potential may then be derived from the discretization of Eq.1 as (Eq.8):

$${v}_{SE} = \frac{l}{2} {\sum }_{n = 1}^{N - 1} {\tilde{Γ}}_{n}^{T} {C}^{Γ} {\tilde{Γ}}_{n}$$

where l corresponds to the initial length of an element, assuming the polyline is uniformly sampled. The strain measure Γₙ may be discretized as proposed by Lang et al. [LLA11] (Eq.9):

$${Γ}_{n} ( {x}_{n} , {x}_{n + 1} , {u}_{n} ) = \frac{1}{l} ( {x}_{n + 1} - {x}_{n} ) - Im ( {u}_{n} {e}_{3} {\overline{u}}_{n} )$$

where the tangent vector is discretized as

$${\partial }_{s} r \approx \frac{1}{l} ( {x}_{n + 1} - {x}_{n} )$$

and only the imaginary part of the quaternion product is considered [KS16].

The discrete bend and twist potential may be derived from the discretization of Eq.3 as (Eq.10):

$${v}_{BT} = \frac{l}{2} {\sum }_{n = 1}^{N - 2} {Ω}_{n}^{T} {C}^{Ω} {Ω}_{n}$$

where Ωₙ may be discretized with the finite quotient expression as proposed by Lang et al. [LLA11] using the quaternion product, denoted by ∘ (Eq.11):

$${Ω}_{n} ( {u}_{n} , {u}_{n + 1} ) = \frac{2}{l} Im ( {u}_{n} ∘ {u}_{n + 1} )$$

Extension of Projective Dynamics with Angular Momentum

In a preferred embodiment, the Projective dynamics (PD) approach as proposed by Bouaziz et al. [BML*14] may be adapted to express the implicit discretized equations for a nodal system by splitting the internal and external forces in the system into a local/global optimization problem. As will be apparent to those skilled in the art, simulating Cosserat rods just with the prior art PD's standard formulation is not possible, as it solely preserves the linear momentum by updating the system's variables with linear velocities and forces. Given that proper modeling of Cosserat rods requires keeping track of body orientations, we propose to overcome this limitation by extending the standard PD formulation to also include the angular momentum term, further comprising for each body discrete element at least an angular velocity, and optionally a torque. With this proposed embodiment, the preservation of the angular momentum becomes a trade-off between all the constraints in the system. Note that we refer to this trade-off as the preservation of the angular momentum for simplicity, but it will be apparent to those skilled in the art that the linear momentum is also preserved in the proposed embodiment.

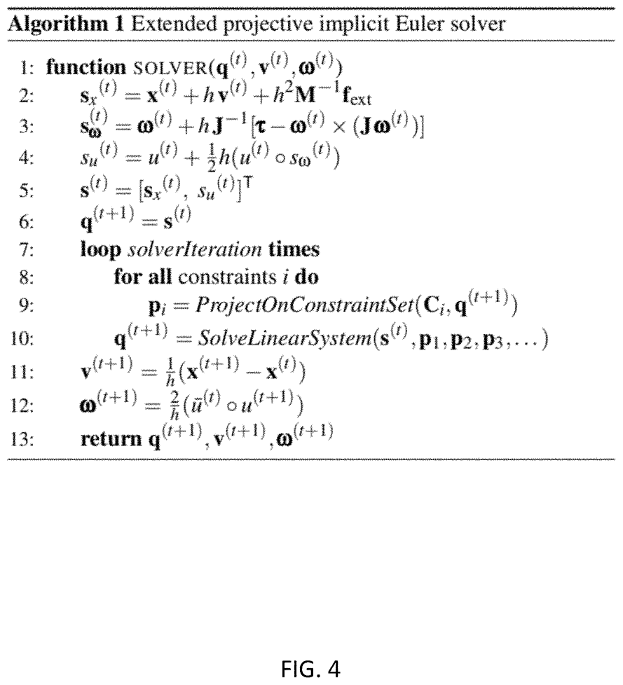

To this end, in a possible embodiment, the moving object's orientations may be incorporated into the solver as additional system variables. As an example, the pseudo-code algorithm of FIG. 4 may be used to extend the projective implicit Euler solver of [BML*14] (Eq.12):

q=[x₁^{T}, . . . ,x_{N}^{T},u₁, . . . ,u_{N-1}]^{T}

where q holds both the positions x∈R³ and the orientations u∈R⁴ (quaternions) for the plurality of elements of the discretized moving object, but other embodiments are also possible. Including orientations as system variables enables to simulate rotational external forces such as torques (τ) using the body's inertia matrices (J) and angular velocities (ω) (see lines 3-4 in the algorithm of FIG. 4).

As the first steps in the algorithm of FIG. 4, both the linear and angular momenta (sₓ^{(t)} and sᵤ^{(t)}, respectively) may be computed with an explicit integration scheme (lines 2-4), where t is the index of the current time step. For the angular momentum, we may first compute s_{ω}^{(t)} as a vector and then use it as the imaginary ω part of a normalized quaternion s_{ω}^{(t)}, whose scalar coefficient is 0 (as proposed for instance in [SM06]); h is the time step size. After the local/global iterative section (lines 7-10), both sets of linear velocities angular v and angular velocities w may updated for the plurality of elements of the discretized moving object (lines 11-12 in the algorithm of FIG. 4) with the new system variables (Eq.13):

$${q}^{( t + 1 )} = {[ {x}^{{( t + 1 )}^{T}} , {u}^{( t + 1 )} ]}^{T}$$

$${v}^{( t + 1 )} = \frac{1}{h} ( {x}^{( t + 1 )} {x}^{( t )} )$$

$${ω}^{( t + 1 )} = \frac{2}{h} ( {\overline{u}}^{( t )} ° {u}^{( t + 1 )} )$$

In a possible embodiment, the angular velocity ω^{(t+1)} may be derived using the temporal derivative for quaternions as proposed for instance in [Wit97], [SM06] (line 12 in the algorithm of FIG. 4).

In a preferred embodiment, the optimization problem may be divided into a local step and a global step.

In the local step, various methods derived for instance from the Position Based methods [MHHR07], where the positions are corrected according to certain desired constraints, may be adapted. In particular, the optimization problem may be solved with regards to a collection of constraints Cᵢ. In one embodiment, as illustrated for instance by line 9 in the algorithm of FIG. 4), the Projective Dynamics solver may be used as defined in Bouaziz et al. [BML*14], but other embodiments are also possible.

In the global step, as illustrated for instance by line 10 in the algorithm of FIG. 4, q^{(t+1)} may be the least squares solution of the linear system (Eq.14):

$$( \frac{{M}^{*}}{{h}^{2}} + {\sum }_{i} {w}_{i} {S}_{i}^{T} {A}_{i}^{T} {A}_{i} {S}_{i} ) {q}^{( t + 1 )} = \frac{{M}^{*}}{{h}^{2}} {s}^{( t )} + {\sum }_{i} {w}_{i} {S}_{i}^{T} {A}_{i}^{T} {B}_{i} {p}_{i}$$

This linear system consists of a sum of potentials: those preserving the linear and angular momenta, represented by s^{(t)}=[sₓ^{(t)},sᵤ^{(t)}]^{T} and those defined per constraint with index i. As will be further detailed throughout this disclosure, the potentials defined per constraint may be derived from the Cosserat constraints, and a weight w, may be assigned per constraint. Similar to the auxiliary variables introduced in [BML*14], the auxiliary projection variables pᵢ may then embed the potential defined per constraint as computed in the local step. Aᵢ, Bᵢ are constant matrices which may be defined per constraint, and S, is the selection matrix, which identifies the variables in q involved in the constraint.

We may further define (Eq.15):

$${M}^{*} = ( \begin{matrix}M & \\ & J\end{matrix} )$$

as the concatenation of M∈R^{3N×3N}, the lumped mass matrix of the points in the discretized object polyline, and J∈R^{4(N-1)×4(N-)}, the inertia matrix of the orientations in the discretized object polyline. J may be defined as the concatenation of

Jn=lρ diag(0,J₁,J₂,J₃),

defined per orientation with index n. l is the distance between orientations, i.e., the length of the segment, ρ is the mass density, and J1, J2, J3 are the moments of inertia from Eq.6 and Eq.7. Note, in Eq.15, the concatenation M* enables the preservation of linear and angular momenta, which is detailed in the following.

Potentials preserving linear and angular momenta may thus be derived with an explicit integration scheme, as illustrated for instance by lines 2-4 in the algorithm 1 of FIG. 4, which we propose to define both for positions and orientations. In a possible embodiment, the angular momentum potential may be derived similar to the calculation of the linear momentum potential in the Projective Dynamics solver as proposed by Bouaziz [BML*14], but using orientations and angular velocities instead of only positions and linear velocities in the formulation. As will be apparent to those skilled in the art of optimization, a potential for preserving the angular momentum may thus be defined by the following optimization problem (Eq.16):

$${min}_{{u}^{( t + 1 )}} \frac{1}{2 {h}^{2}} {‖ {J}^{\frac{1}{2}} ( {u}^{( t + 1 ) } - {s}_{u}^{( t )} ) ‖}_{F}^{2}$$

where the implicitly predicted orientations are (Eq.17):

$${s}_{u}^{( t )} = {u}^{( t )} + \frac{h}{2} ( {u}^{( t )} ∘ {ω}^{( t )} ) + \frac{{h}^{2}}{2} {u}^{( t )} ∘ [ {J}^{- 1} [ τ - {ω}^{( t )} \times J {ω}^{( t )} ) ]$$

This possible embodiment is illustrated by lines 4 and 3 in the algorithm of FIG. 4. Note that when J is multiplied by a vector (e.g., ω) instead of a quaternion (e.g., u), we may take the 3×3 matrix Jₙ′=lρ diag(J1, J2, J3).

### Projective Dynamics Potentials

As defined in Eq.14, the system variables q(t) are updated within the global step according to the momentum potentials and the potentials defined per constraint. In accordance with the Cosserat theory, two potentials may be defined governing the behavior of the rod: the stretch and shear potential on the one hand and the bend and twist potential, on the other hand, discretized in Eq.8 and Eq.10, respectively. We may now formulate the PD constraints and potentials for both these measures and describe how they may be incorporated into the local and global step. Note that in the forthcoming section, we drop the time step superscript (t) for simplicity of reading.

### Stretch and Shear Potential

The stretch and shear constraint C_{SE} minimizes Cosserat theory stretch and shear deformations measured with the stretch strain Γₙ (xₙ, xₙ₊₁, uₙ), defined per rod element with index n (Eq.9). The corresponding stretch and shear potential may then be defined per i-th constraint and minimizes the constraint C_{SE} by (Eq.18):

$${W}_{{SE}_{i}} ( q , {p}_{i} ) = \frac{{w}_{{SE}_{i}}}{2} {‖ {A}_{i} {S}_{i} q - {B}_{i} {p}_{i} ‖}_{F}^{2} + {χ}^{{C}_{SE}} ( {p}_{i} )$$

where Sᵢq=[xₙ₊₁,xₙ,uₙ]^{T} defines the variables involved in the constraint i, selected from q with the matrix Sᵢ. Aᵢ, Bᵢ are constant matrices; and pᵢ are the auxiliary projection variables. The indicator function χ^{CSE}(pᵢ) formalizes the requirement that pᵢ should lie in the constraint manifold C_{SE} and ω_{SE}_{ᵢ} is the potential weight.

The minimization of Eq.18 with regards to the auxiliary projection variables thus leads to the following optimization problem in the local step (Eq.19):

$${min}_{{p}_{i}} {W}_{{SE}_{i}} ( q , {p}_{i} ) = {min}_{{x}_{f}^{*} , {u}_{n}^{*}} {‖ {Γ}_{n} ‖}_{F}^{2}$$

which can be reformulated through the free variables {x_{f}*,uₙ*}.

The free variable

$${x}_{f}^{*} = \frac{1}{l} ( {x}_{n + 1}^{*} - {x}_{n}^{*} )$$

represents the element's differential positions.

Γₙ=x_{f}*−d₃* denotes the Cosserat stretch and shear strain measure. The free variable d₃*=R(uₙ*)e₃ represents the normal of the object's cross-section.

Note that the rotation with uₙ* introduces a non-linear relation between the free variables in Eq.19. Thus, formulating the minimization problem in Eq.19 through the matrices Aᵢ and Bᵢ in Eq.18 is not straightforward. Given that positions and orientations are independent variables, we may decouple Eq.19 into one optimization problem for the positions, i.e. (Eq.20),

$${min}_{{x}_{n + 1}^{*} , {x}_{n}^{*}} {‖ \frac{1}{l} ( {x}_{n + 1}^{*} - {x}_{n}^{*} ) - {d}_{3} ‖}_{F}^{2}$$

and a second optimization problem for the orientations, i.e. (Eq.21)

$${min}_{{u}_{n}^{*}} {‖ {x}_{f} - R ( {u}_{n}^{*} ) {e}_{3} ‖}_{F}^{2}$$

The solution to the minimization problem in Eq.20 is reached when x_{f}*=d₃: the vector x_{f}* is aligned with d₃ and has unit length, i.e. the element's length is the same as in the initial configuration.

Therefore, this optimization problem minimizes the stretch deformation, i.e., the length preservation of the element. The solution to Eq.21 is attained when d₃* is aligned with x_{f}. Hence, the optimal solution of the free variable is uₙ*=uₙ∘∂uₙ, where the orientation uₙ is rotated by ∂uₙ, ∂uₙ being the differential rotation between the vectors d₃ and x_{f}. This optimization problem minimizes the shear deformation, i.e., the rotational difference between the cross-section normal d₃, and the tangent of the element, x_{f} (Eq.9).

In Projective Dynamics [BML*4], the auxiliary projection variables are introduced in the local and global step through the matrices Aᵢ, Bᵢ, as formulated in Eq.18 and Eq.14. For this potential, these matrices are defined as (Eq.22):

$${A}_{i} = [ \begin{matrix}\frac{1}{l} {I}_{3} & - \frac{1}{l} {I}_{3} & {O}_{3 , 4} \\ {O}_{4 , 3} & {O}_{4 , 3} & {I}_{4}\end{matrix} ] , {B}_{i} = [ \begin{matrix}{I}_{3} & {O}_{3 , 4} \\ {O}_{4 , 3} & {I}_{4}\end{matrix} ] , {p}_{i} = [ \begin{matrix}{d}_{3} \\ {u}_{n}^{*}\end{matrix} ]$$

These matrices have two rows, one per each of the minimization problems formulated in Eq.20 and Eq.21; pᵢ embeds the auxiliary projection variables derived in the local solve; Iₖ denotes a k×k identity matrix, and O_{k,m} denotes a k×m zero matrix.

### Bend and Twist Potential

The bend and twist constraint C_{BT} minimizes Cosserat theory bend and twist deformations measured with the twist strain Ωₙ(uₙ,uₙ₊₁), defined per rod element with index n (Eq.11). The corresponding bend and twist potential is defined per i-th constraint and minimizes the constraint C_{BT} by (Eq.23)

$${W}_{{BT}_{i}} ( q , {p}_{i} ) = \frac{{w}_{{BT}_{i}}}{2} {‖ {A}_{i} {S}_{i} q - {B}_{i} {p}_{i} ‖}_{F}^{2} + {χ}^{{C}_{BT}} ( {p}_{i} )$$

where Sᵢq=[uₙ,uₙ₊₁,]^{T} are the variables involved in the constraint, the adjacent quaternions, which are selected from q with the selection matrix Sᵢ, and pᵢ are the auxiliary projection variables.

The minimization of Eq.23 with regards to the auxiliary projection variables leads to the following optimization problem in the local step (Eq.24):

$${min}_{{p}_{i}} {W}_{{BT}_{i}} = {min}_{{u}_{n}^{*} , {u}_{n + 1}^{*}} {‖ {Ω}_{n} ‖}_{F}^{2}$$

which can be reformulated through the free variables {uₙ*,uₙ₊₁*}. Ωₙ denotes the curvature vector, i.e., the relative curvature between adjacent quaternions. The solution to the minimization problem is reached when the relative curvature Ωₙ between the adjacent orientations is 0. The optimal solution to Eq.24 may then be derived as (Eq.25):

$${u}_{n}^{*} = {u}_{n} ∘ \frac{{Ω}_{n}}{2} and {u}_{n + 1}^{*} = {u}_{n + 1} ∘ \frac{{\overline{Ω}}_{n}}{2}$$

where ∘ denotes a quaternion product. The current orientations uₙ and uₙ₊₁ are rotated with halfway of the curvature vector and its conjugate, respectively. With this solution, the resulting orientations uₙ* and uₙ₊₁* have the same direction, minimizing the relative curvature Ωₙ=0 between them.

In this formulation, the curvature vector is defined as Ωₙ=Im(uₙ∘uₙ₊₁). This expression does not need to be scaled by 2/l, as opposed to Eq.11, given that scaling a minimization problem by a scalar leads to the same result. Instead, the potential in Eq.23 may be scaled by ω_{BTi}, using the potential weight formulation as will be further discussed in more detail in the next section. In Projective Dynamics [BML*14], the auxiliary projection variables are introduced in the local and global step through the matrices Aᵢ, Bᵢ, as formulated in Eq.23 and Eq.13. For this potential, these matrices are defined as Aᵢ=Bᵢ=I₈, where Iₖ is a k×k identity matrix.

### Potential Weight Formulation

The potential formulations in the discretized Cosserat theory (v_{SE},v_{BT}) in Eq.8 and Eq.10 are defined by the product of the strain measures (Γₙ,Ωₙ) with certain weight matrices (C^{Γ},C^{Ω}). In a preferred embodiment, the weight matrices may depend on some material parameters such as the Young's modulus E or the radius r of the rod. Additionally, the discrete strain measures may be scaled by the length of the segment l.

In a possible embodiment, the Projective Dynamics's potential weights such that the potentials may be formulated equivalent to the ones formulated in Cosserat theory. This enables to compare our simulations to a reference solution generated with finite differences, which is parametrized with variables such as E, r or the mass density.

For the stretch and shear potential, the weight ω_{SE}, in Eq.18 may be formulated as (Eq.26):

w_{SE}_{ᵢ}=EA₃l

where A₃=πr² is the area of the cross-section. In this formulation, we may assume that the three components on the weight matrix C_{Γ} are scaled by the constant E, i.e., Young's modulus, as opposed to the formulation in Eq.5, where some of the components of the matrix are instead scaled by the shear modulus G. With this assumption, we may neglect the Poisson ratio v in this weight.

The reason for assuming a uniform scaling is that in Projective Dynamics, potentials are defined as the Frobenius norm of a certain deformation (Eq.18). In our potentials, the deformations are vectors. Its Frobenius norm is a scalar, and hence the weight ω_{SEi} in Eq.18 needs to be a scalar as well.

Our formulation of the weight may be additionally scaled by the constant l, given that the expression of the discretized potential is also proportional to this constant (Eq.8).

For the bend and twist potential, the weight ω_{BTi} in Eq. (23) may be formulated as (Eq.27):

$${w}_{{BT}_{i}} = \frac{4 {GJ}_{3}}{l}$$

where

$${J}_{3} = \frac{π {r}^{4}}{2}$$

is me expression or me polar moment of inertia (Eq.6). In this formulation we may assume again a constant scaling, by the shear modulus

$$G = \frac{E}{2 ( 1 + γ )} ,$$

γ being the Poisson ratio.

The strain measure Ωₙ may be scaled by 2/l (Eq.11). The potential γ_{BTi} in Eq.10 is defined by the product 1 Ωₙ^{T}C^{Ω}Ωₙ which leads to the formulated weight

$$ω {BT}_{i} = l \frac{2}{l} ( G {J}_{3} ) \frac{2}{l} ,$$

and therefore to the simplified expression in Eq.27.

Note that the formulation of ω_{BTi} is divided by 1, as opposed to the formulation of ω_{SEi}. We observed that the formulation of these potential weights ensures mesh independence in our simulations within a few iterations. Experiments also show that modifying the weights affects the convergence rate of the solver. In practice, when weighted as described above, with already 2 to 10 iterations the system gives similar results to finite element methods.

### Experimental Results

Various simulations have been conducted for different rod parameters, r as the radius (mm), ρ as the mass density (g/m³), E as the Young's modulus (MPa), L as the length (m), h as the time step (ms) and −g_{y} as the gravity (m/s²), as listed in Table 1:

TABLE 1
Parameters settings from representative experiments EXP1 to EXP7.

| PARAM- | | | | | | | |
|---|---|---|---|---|---|---|---|
| ETERS | EXP1 | EXP2 | EXP3 | EXP4 | EXP5 | EXP6 | EXP7 |
| r | 3 | 8 | 3 | 3 | {2,6,8} | 3 | 1.5 |
| P | 1.3 | 4.3 | 1.3 | 1.3 | 1.3 | 1.3 | 1.3 |
| E | 1 | 1 | 1 | 1 | {1,50} | 500 | 100 |
| L | 1 | 0.5 | 1 | 1 | 1 | 0.2 | 0.7 |
| h | 1 | 1 | 1 | 1 | 1 | 1 | 10 |
| g_{y} | 9.861 | 9.861 | 9.861 | 0 | 9.861 | 0 | 9.861 |

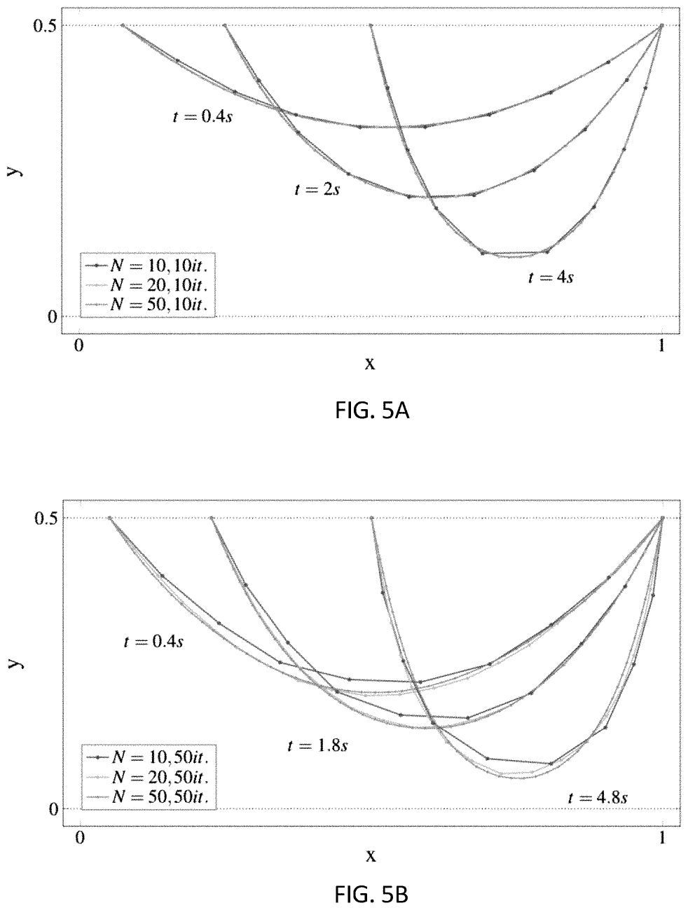

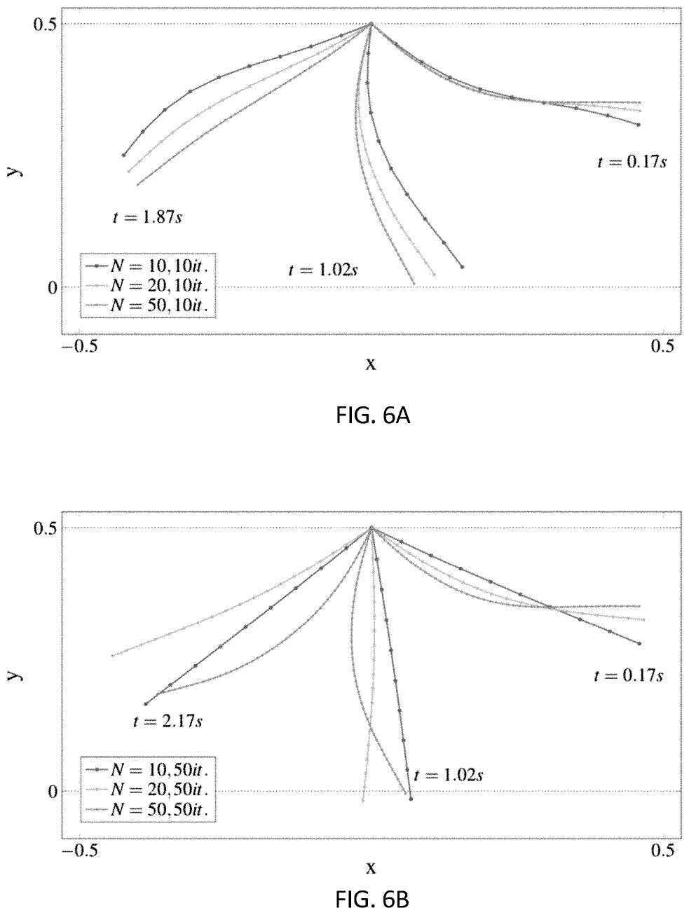

FIG. 5A-8B illustrate the results of the first experiment (EXP1) at three time snapshots of motion simulations of a hanging rod, under the gravitational force and for different mesh resolutions. The two endpoints of the rod are attached: the right endpoint is fixed, while the left endpoint is gradually moved in parallel to the x-axis. FIG. 5A shows the results with the proposed method using the Projective Dynamics solver adapted from Bouaziz et al. [BML*14] to preserve the angular momentum. FIG. 5B shows the results with the prior art PBD method of [KS16] as implemented by Bender [Ben]. The number of iterations in the PBD method are adjusted such that it has the same computation time as the PD simulation. Note, in PBD, the number of iterations affects the material properties, i.e., the higher the number of iterations, the stiffer the material gets. Thus, when using PBD, we use a constant number of iterations when comparing different resolutions. Although PBD presents similar motion for the different resolutions, PD converges faster to a mesh independent solution, by applying only 10 iterations. This improvement is even more apparent with the second experiment (EXP2) as illustrated by FIGS. 6A and 6B, showing the motion simulation at three time snapshots for a hanging rod with only one endpoint attached, under the gravitational force and for different mesh resolutions. FIG. 6A shows the results with the proposed method using the Projective Dynamics solver adapted from Bouaziz et al. [BML*14] to preserve the angular momentum. FIG. 6B shows the results with the prior art PBD method of [KS16] as implemented by Bender [Ben]. The number of iterations in the PBD method are adjusted such that it has the same computation time as the PD simulation. The proposed PD method shows (FIG. 6A) the same characteristic motion for different mesh resolutions by only applying 10 iterations, while with the prior art PBD method (FIG. 6B), after the same computation time, the rod dynamics in FIG. 8B are still not equivalent. Within this test, we demonstrate how a possible implementation of the proposed method converges faster, in terms of computation time, to a mesh independent solution, compared to recent prior art PBD methods and implementations.

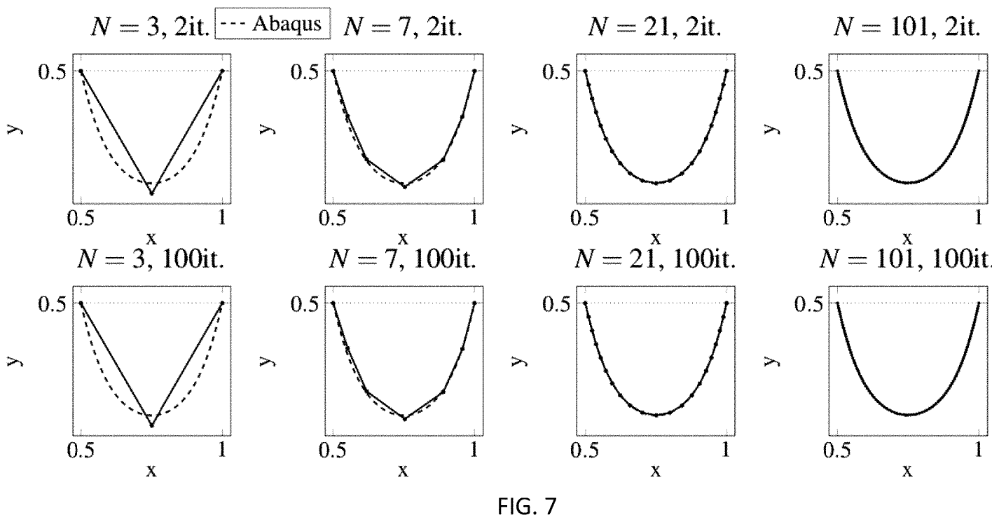

FIG. 7, FIG. 8A, FIG. 8B and FIG. 9 illustrate the results of further experiments (EXP3, EXP4, EXP5) in which the results of the proposed method using the Projective Dynamics solver adapted from Bouaziz et al. [BML*14] to preserve the angular momentum are compared with the results of a high-resolution FEM simulation using the Abaqus software [HS02], where the rod is discretized with B21 Timoshenko beams. A Cosserat rod can be considered as the geometrically nonlinear generalization of a Timoshenko rod, allowing to model extension and shearing apart from bending and twist, as opposed to Kirchhoff rods. All the simulations in Abaqus are implemented with the explicit procedure using 990 elements. The equations of motion are integrated using the explicit central difference integration rule (Abaqus 6.14 Theory Guide, Section 2.4.5). The time step is defined automatically by the software according to the stability conditions of each simulation. Since the equations of motion used in our model are solved differently than the ones used in the reference Abaqus solution, comparisons of simulations are performed in static equilibrium, which is reached when the rods do not undergo further motion.

Simulations with a small Young's modulus and a small radius (Table 1, EXP3 and EXP4) result in a highly elastic behavior. Such elastic materials present high frequency vibrations in the Abaqus simulations, which we damped with the bulk viscosity parameter (we used Linear bulk viscosity parameter=0.07, Quadratic bulk viscosity parameter=1.3). Additionally, we damped the motion with the a dampening coefficient in the material properties (we used α=0.8). Thus, damping is an issue when comparing to a reference solution. The damping models used in both methods are different and therefore an exact position correspondence in both simulations is difficult to achieve. For this reason, in order to compute the convergence of our simulation in respect to a reference solution, the rod's potential may be used as an error measure, instead of taking the positional difference. The convergence of the proposed method towards the results of the reference high-resolution FEM method is demonstrated by FIG. 7 and FIGS. 8A and 8B for different resolutions and different number of iterations. In FIGS. 8A and 8B, the static equilibrium scenario assumes a 360° twist applied on the endpoint orientation u_{N-1}. FIG. 8A shows the top-down view of different resolution twisted rods converging towards the Abaqus reference solution. FIG. 8B shows a side view of different resolution twisted rods converging towards the Abaqus reference solution. FIG. 9 further shows the comparison of the bending behavior for a simulation with the proposed method and the FEM reference solution (Abaqus) for different geometric and material parameters. Different widths of the rod representative lines here denote different rod radii r, each combined with different values of Young's modulus E (Table 1—EXP5). For this simulation, the rod is initialized along the x-axis with two attached endpoints at x₁=0, x_{N}=L, under the effect of the gravitational force. The endpoint x₁ is displaced until x₁=L.

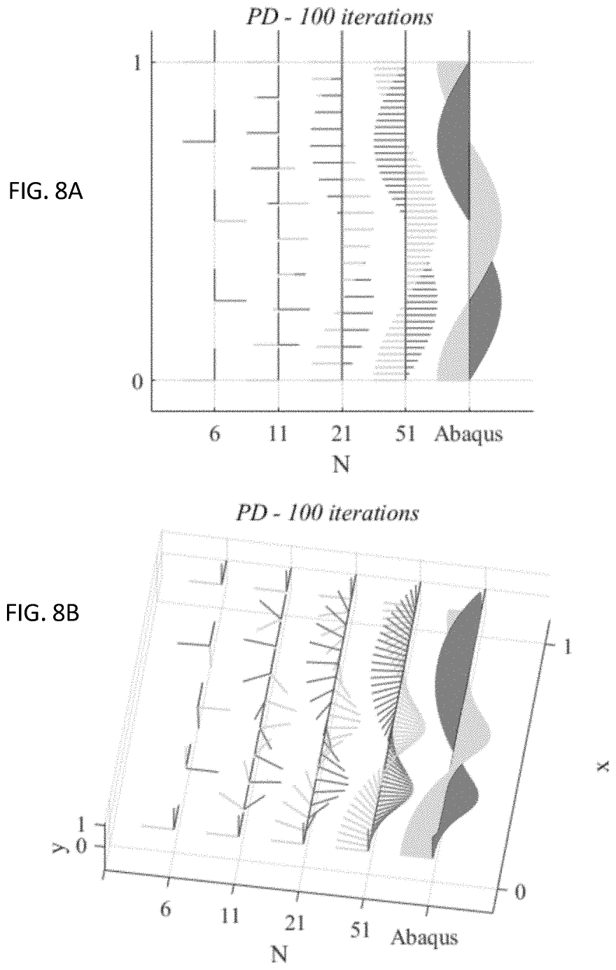

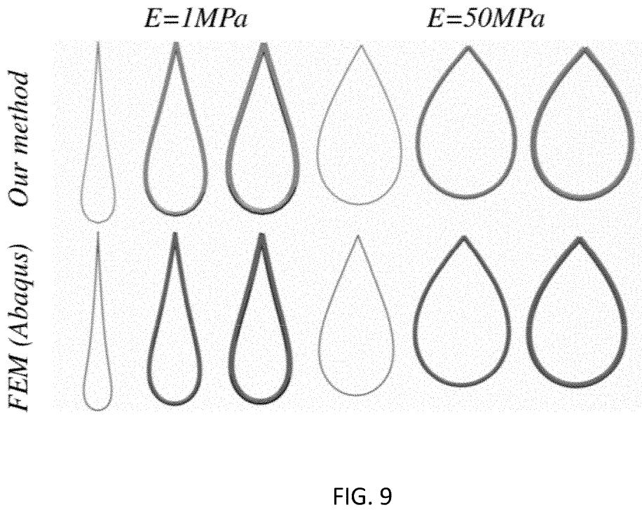

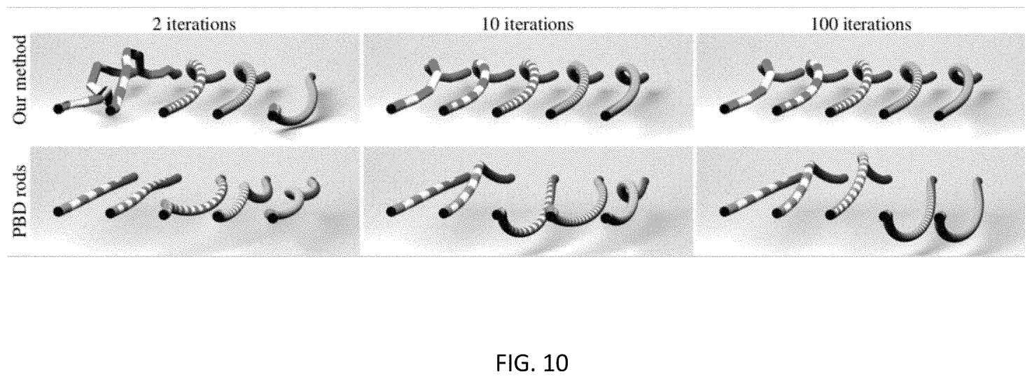

FIG. 10 further illustrate the results of an out-of-plane curl effect simulation experiment (EXP6) in which the results of the proposed method using the Projective Dynamics solver adapted from Bouaziz et al. [BML*14] to preserve the angular momentum are compared with the results of the PBD method of [KS16]. Twisted and compressed rods are simulated at different mesh resolutions. This simulation validates both constraints at the same time, as opposed to the separate analysis from previous experiments. The rod is initialized along the x-axis, with both endpoints x₁=0 and x_{N}=L attached. Twists of 120° and −120° are applied gradually to the orientation frames u₁ and u_{N-1}, respectively. After some frames, the rod is slightly compressed by displacing both endpoints gradually towards the center of the rod. This results in an out-of-plane curl, a common feature in rod simulation [vdHT00]. The same experiment is executed with {2, 10, 100} iterations and different mesh resolutions N={10, 20, 50, 100, 200}. As opposed to the PBD prior art method, from 10 iterations onward, the proposed method converges to a realistic simulation of the curls, no matter which resolution is used (mesh independent solution).

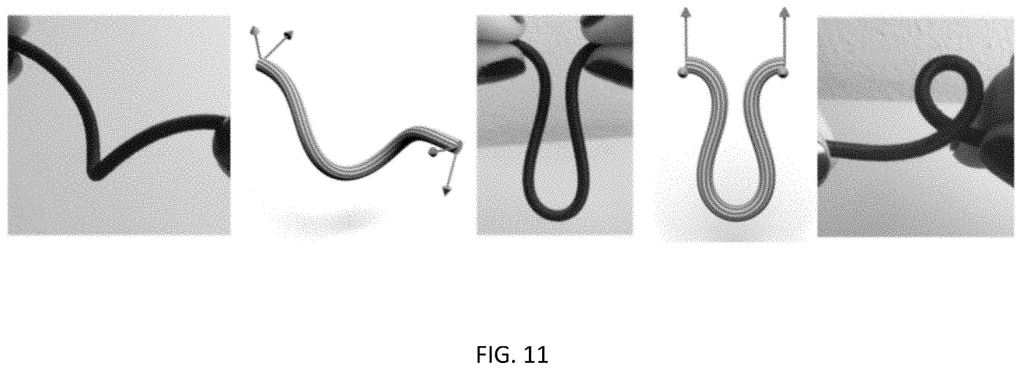

FIG. 11 shows a further experiment comparing a real rod to the simulations with the proposed method using the Projective Dynamics solver adapted from Bouaziz et al. [BML*14] to preserve the angular momentum, using the same geometric and material parameters (Table 1—EXP7). This further demonstrates the realism which may be achieved with the proposed method.

Further experiments (not illustrated) have demonstrated the robustness of the proposed method compared to prior art methods for various deformation simulations. The proposed formulation also inherently supports the detection and response to self-collisions, when adapted from the edge-edge distance constraint as in the prior art PBD method implementation of [Ben]. The rod tangles with itself after applying torsional deformation on the endpoints, leading to the formation of plectonemes. Interactive displacement of the endpoints enables the formation of knots.

As will be apparent to those skilled in the art, the computer graphics generator system 100 may also be part of a high-quality image generation system for realistically simulating bending and twisting rods such as ropes, cables, threads, nets, voluble plants, human hair or animal fur, etc. The proposed methods as described in the present disclosure may then be implemented by the computer graphics generator system 100 as a computationally more efficient and more robust alternative to the rod simulation methods described for instance in US2010156902 by ETRI or in US20170169136 by Autodesk, for instance as extensions to commercially available high-end 3D computer graphics and 3D modelling software packages such as AUTODESK MAYA® or 3Ds STUDIO MAX®.

### Surgical Threading Training Simulator

In a possible embodiment for a surgical simulator, 3D object bodies comprise anatomy tissue models 115 (for instance, a knee ligament model) as well as rod models 125 (for instance, a suture thread model for repairing a torn knee ligament). All models are registered in a storage unit of the surgical simulator with predetermined physical properties as object parameters that are specific to each object model. During the surgical simulator real-time operation, position and orientation information of the end-user and/or of an instrument manipulated by the end-user may be tracked by sensors and combined with graphical models of 3D object bodies 115, taking into account their physical properties to realistically render virtual events such as collisions between the virtual instrument and a simulated anatomy tissue. In the case of a suture simulation, the surgical simulator further takes into account a set of additional constraints in accordance with the material and geometrical properties of the suture rod models 125 to simulate and render a computer-generated scene comprising the simulated moving instrument, the simulated anatomy tissue, and the simulated rod model around one or more virtual suture fixations between the simulated anatomy tissue and the simulated rod.

In a possible embodiment, the computer graphics generator system 100 may be part of the surgical simulation system as described in U.S. Pat. No. 8,992,230, the description of which is hereby incorporated by reference in its entirety. In such a simulator, the computer graphics generator system 100 acquires in real-time from tracking sensors the position and orientation of the needle holder instrument as well as the position and orientation of the endoscope replica instrument as manipulated by the end user (105).

The computer graphics generator system 100 determines with a constraint builder module (not represented) the set of constraints applicable to the virtual surgical thread, represented as a rod attached on one end to the needle. In particular, in the case of a suturing simulation, the computer graphics generator system 100 determines collisions between the anatomy tissue model 115 and the needle model corresponding to the needle held by the needle holder instrument to create and realistically render suture fixations (where the suture pierces the anatomy tissue) in the displayed scene. The virtual reality scene therefore depends on the tracked motion of the needle holder, the detected collision points in the virtual environment, the geometrical and material properties of the rod, and the physical properties of the anatomy tissue models to be sutured in the virtual environment simulation. The computer graphics generator system 100 accordingly calculates, with a local-global solver unit, the simulated suture fixation points, the simulated anatomy tissue models, and the simulated thread (rod model) to calculate the virtual reality scene and to render it onto the surgical simulator display. Preferably, the local-global solver implements the proposed adaptation of the Projective Dynamics solver as described herein, but other embodiments are also possible. As will be apparent to those skilled in the art, to simulate the displacement and possible deformations of the anatomy tissue model due to the collision at the suture fixation point, when the Projective Dynamics method of Bouaziz et al. [BML*14] is applied, the computer graphics generator 100 may calculate area and volume-preservation and/or strain constraints from the material properties of the anatomy tissue model 115, but other embodiments are also possible. However, as will be apparent to those skilled it the art, neither Bouaziz et al. [BML*14] or other prior art methods such as the Position-Based Dynamics solver of [KS16] provide means for efficient and realistic rendering of a twisting and bending surgical thread in relation with the computer graphics simulation of the anatomy tissue models 115 at the suture fixation points associated with a suturing training scenario. In a preferred embodiment, this limitation of the prior art methods is addressed by additional modeling of the surgical thread as a Cosserat rod with predetermined geometrical and material properties.

A suture fixation point may be first initialized both on the suture surgical thread and the anatomy tissue by the computer graphics generator system 100 when it detects a collision between the suturing needle and the tissue, where the suture fixation is initialized. The computer graphics generator system 100 may accordingly generate, at the suture fixation point, a suture fixation constraint for the suture as well as a positional constraint for the anatomy tissue model. Thus, if the anatomy tissue deforms, the suture surgical thread remains attached to the suture fixation location. In particular, if the user pulls from one of the suture endpoints, the suture will slide through the suture fixation location, thus simulating a needle eye.

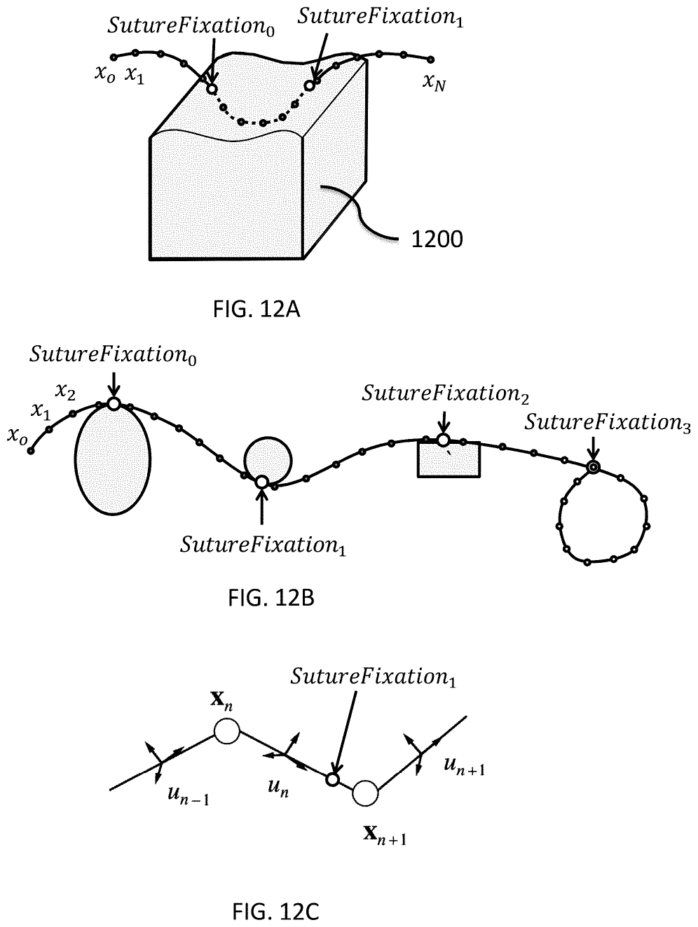

FIG. 12A represents an abstract 3D view of a surgical thread modeled as a Cosserat rod discretized into a series of Cosserat elements [x₀,x₁], [x₁,x₂] . . . [x_{N-1},x_{N}]. The rod may be attached to an anatomy model element 1200, for instance a ligament, at one or more suturing fixation points. Accordingly, in the rendering of the final suturing scene, the deformation of the anatomy model and thus the motion of the rod initiated by the motion of the needle holder attached on the rod endpoint x₀ depend on the one hand on the geometrical and material properties of the rod model and on the other hand on the material properties of the anatomy tissue model. FIG. 12B represents an abstract 2D view of a similar rod, which ends on its other extremity with a loop. More complex geometries may also be similarly modeled, for instance to simulate the tying of a knot in the virtual environment.

As illustrated in FIG. 12C, a suture fixation may be found at any placement along a Cosserat element segment [xₙ,xₙ₊₁]. In a possible embodiment, at least one of the suture fixation elementary constraints may be parametrized using barycentric coordinates according to the placement of the suture fixation along the Cosserat rod element, but other embodiments are also possible.

### Advantages of the Proposed Methods Over the Prior Art

As will be apparent to those skilled in the art, the proposed methods bring several improvements over the prior art methods. Compared to the FEM methods and/or mass spring models such as those of U.S. Pat. No. 8,956,165, the proposed methods are faster to compute, more stable and enable more realistic rendering. Compared to the Projective Dynamics (PD) solver of [BML*14], by adding orientation variables to enable the simulation of surgical threads as Cosserat rods, and by adding material properties of the rods to the PD solver constraints, it is possible to simulate much more realistically complex surgical training scenarios such as suturing. Compared to the Position Based Dynamics (PBD) solver of [KS16], the addition of material properties of the rods to the solver constraints results in a much more realistic simulation for a diversity of scenario and in particular virtual suturing.

### Other Embodiments

While the proposed methods and systems have been primarily described for simulation of rods, it will be apparent to those skilled in the art that they may also more generally apply to the computer-implemented simulation of other moving objects with a changing orientation, which may be either rigid or deformational. In particular, they may also apply to any twisting and bending Cosserat thin objects, such as Cosserat shells and Cosserat volume primitives.

While the proposed methods and systems have been primarily described and experimented with an adaptation of the Projective Dynamics solver developed by Bouaziz et al. [BML*14] to preserve the angular momentum, it will be apparent to those skilled in the art that other PD solvers may be similarly adapted. Examples of solvers which may be adapted to preserve the angular momentum with Cosserat objects while enabling a computationally efficient parallel implementation include, but are not limited to, the solvers recently disclosed by Wang [Wan15] or Fratarcangeli et al. [FTP16].

Further possible embodiments include the use of a multi-grid rod representation to speed-up the rod simulation. Alternately, local refinements may be applied to regions of interest which require a higher resolution, such as regions where the knots are being tied.

Those skilled in the art will appreciate other variations to the disclosed embodiments but comprised by the appended claims from practicing the claimed disclosure and/or from a study of the description, drawings and claims. In the claims, the word “comprising” does not exclude other elements or steps, and the indefinite article “a” or “an” does not exclude a plurality. A single processor or other digital processing unit may fulfil the functions of several items recited in the claims and features recited in mutually different dependent claims may be combined. Reference signs in the claims, if any, are provided for illustrative purposes only.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, a processor being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an application program interface (API)).

The performance of certain of the operations may be distributed among the one or more processors. The processors may be residing within a single machine, such as a Graphical Processing Unit (GPU). They may also be deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Although an overview of the inventive subject matter has been described with reference to specific example embodiments, various modifications and changes may be made to these embodiments without departing from the broader spirit and scope of embodiments of the present invention. Such embodiments of the inventive subject matter may be referred to herein, individually or collectively, by the term “invention” merely for convenience and without intending to voluntarily limit the scope of this application to any single invention or inventive concept if more than one is, in fact, disclosed.

The embodiments illustrated herein are described in sufficient detail to enable those skilled in the art to practice the teachings disclosed. Other embodiments may be used and derived therefrom, such that structural, mathematical and logical substitutions, formulations and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, formulations, or structures described herein as a single instance. Additionally, boundaries between various resources, operations, formulations, modules, engines, mathematical solvers, and data stores are somewhat arbitrary, and particular operations are illustrated in a context of specific illustrative configurations. Other allocations of functionality are envisioned and may fall within a scope of various embodiments of the present invention. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present invention as represented by the appended claims. The specification and drawings are, accordingly, to be regarded in an illustrative rather than a restrictive sense.

### REFERENCES

[BAV*10] BERGOU M., AUDOLY B., VOUGA E., WARDETZKY M., GRINSPUN E.: Discrete Viscous Threads. ACM Trans. Graph. (Siggraph) (2010).
[Ben] BENDER J Position Based Dynamics Online Implementation. https://github.com/InteractiveComputerGraphics/PositionBasedDynamics/. Accessed: 2017-09-17.
[BML*14] BOUAZIZ S., MARTIN S., LIU T., KAVAN L., PAULY M.: Projective Dynamics: Fusing Constraint Projections for Fast Simulation. ACM Trans. Graph. (Siggraph) (2014).
[BWR*08] BERGOU M., WARDETZKY M., ROBINSON S., AUDOLY B., GRINSPUN E.: Discrete Elastic Rods. ACM Trans. Graph. (Sig-graph) (2008).
[CBD13] CASATI R., BERTAILS-DESCOUBES F.: Super Space Clothoids. ACM Trans. Graph. (Siggraph) (2013).
[Di192] DILL E. H.: Kirchhoff's Theory of Rods. Archive for History of Exact Sciences (1992).
[DKWB18] DEUL C., KUGELSTADT T., WEILER M., BENDER J.: Di-rect Position-Based Solver for Stiff Rods. Computer Graphics Forum (Eurographics) (2018).
[FTP16] FRATARCANGELI M., TIBALDO V., PELLACINI F.: Vivace: A practical gauss-seidel method for stable soft body dynamics. ACM Trans. Graph. (Siggraph) (2016).
[HS02] HIBBITT K, SORENSEN: ABAQUS/CAE User's Manual. Hib-bitt, Karlsson & Sorensen, Incorporated, 2002.
[Kir59] KIRCHHOFF G.: Uber das Gleichgewicht and die Bewegung eines unendlich diinnen elastischen Stabes. Journal für die Reine and Angewandte Mathematik (1859).
[KS16] KUGELSTADT T., SCHOEMER E.: Position and Orientation Based Cosserat Rods. Eurographics/SIGGRAPH Symposium on Computer Animation (2016).
[LLA11] LANG H., LINN J., ARNOLD M.: Multi-Body Dynamics Sim-ulation of Geometrically Exact Cosserat rods. Multibody System Dynamics (2011).
[MHHR07] MÜLLER M., HEIDELBERGER B., HENNIX M., RATCLIFF J.: Position Based Dynamics. Journal of Visual Communication and Image Representation (2007).
[MMC16] MACKLIN M., MÜLLER M., CHENTANEZ N.: XPBD: Position-based Simulation of Compliant Constrained Dynamics. Proceedings of ACM Motion in Games (2016).
[Nak] NAKAGAWA N.: XPBD Online Implementation. https://github.com/nobuo-nakagawa/xpbd/. Accessed: 2018-03-28.
[Pai02] PAI D. K.: STRANDS: Interactive Simulation of Thin Solids using Cosserat Models. Computer Graphics Forum (2002).
[Rub13] RUBIN M. B.: Cosserat Theories: Shells, Rods and Points. Springer Science & Business Media, 2013.
[Sim85] SIMO J.: A Finite Strain Beam Formulation. The Three-dimensional Dynamic Problem. Part I. Computer Methods in Applied Mechanics and Engineering (1985).
[SM06] SCHWAB A. L., MEIJAARD J.: How to Draw Euler Angles and Utilize Euler Parameters. Proceedings of IDETC/CIE (2006). 4
[ST07] SPILLMANN J., TESCHNER M.: CORDE: Cosserat Rod Elements for the Dynamic Simulation of One-Dimensional Elastic Objects. Eurographics/SIGGRAPH Symposium on Computer Animation (2007).
[USS14] UMETANI N., SCHMIDT R., STAM J.: Position-based Elastic Rods. Eurographics/SIGGRAPH Symposium on Computer Animation (2014).
[vdHT00] VAN DER HEIJDEN G., THOMPSON J.: Helical and Localised Buckling in Twisted Rods: A Unified Analysis of the Symmetric Case. Nonlinear Dynamics (2000).
[Wan15] WANG H.: A Chebyshev Semi-iterative Approach for Accelerating Projective and Position-based Dynamics. ACM Trans. Graph. (Siggraph) (2015).
[Wit97] WITKIN A.: Physically Based Modeling: Principles and Practice—Rigid Body Dynamics, Lecture Notes I. Computer Graphics (1997).

1. A computer graphics method for rendering, with a processor, a twisting and bending surgical thread in a medical simulator, the method comprising:
acquiring in real time a position and an orientation of a needle holder instrument replica and a position and an orientation of an endoscope replica from one or more sensors tracking the manipulation of an instrument and an endoscope by an end user;
detecting, from the tracked position and orientation of the needle holder, a collision between a simulated needle and an anatomy tissue model in a virtual environment;
creating, at the collision, a suture fixation point in the virtual environment;
determining a set of constraints applicable to a virtual surgical thread at the suture fixation point, wherein the virtual surgical thread is represented in the virtual environment as a rod attached to the anatomy tissue model at the suture fixation point, wherein the set of constraints comprise at least:
a bend and twist constraint which depends on geometrical and material properties of the rod model; and
a suture fixation constraint which depends on physical properties of the anatomy tissue model at the suture fixation point, to constrain the suture to slide through the suture fixation point in response to the tracked position and orientation of the needle holder indicating that the user is pulling the suture from the anatomy tissue model at the suture fixation point;
discretizing and modelling the rod as a Cosserat rod with one or more rod elements;
estimating, with a local-global solver, the position and orientation of each rod element using the set of constraints;
determining, from the position and orientation of the needle holder instrument replica and the position and orientation of the endoscope replica, which elements of the rod are visible in the scene to be rendered; and
rendering each visible rod element in the computer graphics simulation.

2. The method of claim 1, wherein estimating, with a local-global solver, the position and orientation of each rod element using the set of constraints comprises:
identifying, for each of the Cosserat rod elements, a current position and orientation, and a current linear velocity and a current angular velocity corresponding to bending or twisting motion to simulate;
estimating, for each of the Cosserat rod elements, a predicted position and orientation. as a function of the current position, orientation, linear velocity and angular velocity;
formulating, for each of the plurality of elements, object motion constraints Cᵢ corresponding to the rod bending or twisting motion to simulate, the rod motion constraints Cᵢ comprising a Cosserat rod bend and twist constraint C_{BT} as a function of a twist strain defined for each rod element;
for each of the plurality of Cosserat rod elements, projecting, with a local solver, the predicted position and orientation into auxiliary projection variables pᵢ on the rod motion constraints Cᵢ, wherein the local solver projection on the Cosserat rod bend and twist constraint C_{BT} is optimized when the relative curvature between any pair of adjacent element orientations is zero;
estimating, with a global linear system solver, a refined predicted position and a refined predicted orientation for each of the plurality of elements of the bending or twisting rod as a function of the auxiliary projection variables pᵢ for each of the plurality of elements and of the set of constraints.

3. The method of claim 2, wherein the local and global solver steps are iterated a plurality of times.

4. The method of claim 2, wherein the Cosserat rod elements are associated with discretized position variables x∈R³ and discretized Cosserat rod orientation quaternion variables u∈R⁴.

5. The method of claim 2, wherein the object motion constraints Cᵢ further comprise a Cosserat rod stretch and shear constraint C_{SE} as a function of a stretch strain defined for each rod element.

6. The method of claim 5, wherein the local solver projection on the Cosserat rod stretch and shear constraint C_{SE} comprises a first local optimization step, with the local solver, on the position variables and a second local optimization step, with the local solver, on the orientation variables, the first and the second steps being independent from each other.

7. The method of claim 6, wherein the solution to the first local optimization on the position variables is reached when the element's differential positions have a unit length and are aligned with the normal of the Cosserat rod's cross section, so as to preserve the element's length as in its initial configuration.

8. The method of claim 6, wherein the solution to the second local optimization on the orientation variables is reached when the rotational difference between the normal of the Cosserat rod's cross section and the tangent of the element is minimal.

9. The method of claim 2, wherein predicting, with a global solver, the motion of the Cosserat rod comprises calculating a matrix of weighted Cosserat potentials, the potentials being calculated as a function of the auxiliary projection variables pᵢ for the plurality of elements, and the weights being calculated as a function of the material and the geometrical properties of the Cosserat rod.

10. The method of claim 9, wherein the geometrical properties of the Cosserat rod are defined by at least one of the radius of the rod and the length of the rod.

11. The method of claim 9, wherein the material properties of the Cosserat rod are defined by at least a mass density of the rod material.

12. The method of claim 9, wherein the rod is elastic and the material properties of the Cosserat rod are further defined by the Young's modulus of the rod.

13. The method of claim 1, further comprising calculating, for each of the plurality of elements, a predicted linear velocity as a function of the current position and the refined predicted position for each element, and a predicted angular velocity as a function of the current orientation and the refined predicted orientation for each element; and using the predicted linear velocity, the predicted angular velocity and the predicted refined predicted position and orientation as the current estimates for each element of the twisting or bending rod in a next rendering calculation.

14. The method of claim 1, wherein the twisting or bending rod is any object selected from the group consisting of a surgical thread, a suturing thread, a blood vessel, and a strand of hair.

15. The method of claim 1, wherein the suture fixation constraint is formulated to minimize a set of elementary constraints such as a constraint on the position of the suture fixation, a constraint on the orientation of the suture fixation, a constraint to ensure that the suture preserves its rest length, and/or a pulling constraint on the fixation tissue using the suture's position at the suture fixation as target location from the pulling.

16. The method of claim 15, wherein at least one of the suture fixation elementary constraints is parametrized using barycentric coordinates according to the placement of the suture fixation along the Cosserat rod element.
